(19) United States
(12) Patent Application Publication
(10) Pub. No.: US 2008/0268049 A1
(43) Pub. Date: Oct. 30, 2008

(54) Stable Solid Dosage Forms of Amlodipine and Benazepril

(76) Inventors: Mona Dhaliwal, Uttar Pradesh (IN); V.V. Ram Mohan Rao, Andhra Pradesh (IN); Vishnubhotla Nagaprasad, Andhra Pradesh (IN)

Correspondence Address: RANBAXY INC., 600 COLLEGE ROAD EAST, SUITE 2100, PRINCETON, NJ, 08540, US

(21) Appl. No.: 11/816,034

(22) PCT Filed: Feb. 10, 2006
(86) PCT No.: PCT/IB06/00276

§ 371 Date: Jun. 6, 2008

(30) Foreign Application Priority Data
Feb. 11, 2005 (IN) 304/DEL/2005

(51) Int. Cl. A61K 31/55; A61K 9/20; A61P 9/12

(52) U.S. Cl. 424/474; 514/212.07

(57) ABSTRACT

The technical field of the invention relates to stable solid dosage forms of amlodipine besylate and benazepril hydrochloride; and processes for their preparation. In particular, the solid dosage forms having reduced levels of 3-ethyl methyl [(2-aminoethoxy)methyl](2-chlorophenyl)methylpyridine-3,5 dicarboxylate (“impurity D”) and total impurities when free of dicalcium phosphate.

### FIELD OF THE INVENTION

The technical field of the invention relates to stable solid dosage forms of amlodipine besylate and benazepril hydrochloride; and processes for their preparation. In particular, the solid dosage forms having reduced levels of 3-ethyl methyl [(2-aminoethoxy)methyl] (2-chlorophenyl)methylpyridine-3,5 dicarboxylate (“impurity D”) and total impurities when free of dicalcium phosphate.

### BACKGROUND OF THE INVENTION

Amlodipine is a long acting calcium channel blocker marketed by Pfizer as amlodipine besylate under the trade name Norvasc®. It is available as oral tablets in strengths of 2.5 mg, 5 mg, and 10 mg, and is indicated for the treatment of hypertension, chronic stable angina and vasospastic angina. The inactive ingredients in the Norvasc® tablets include microcrystalline cellulose, dibasic calcium phosphate anhydrous, sodium starch glycolate, and magnesium stearate.

The preparation of amlodipine base is described in U.S. Pat. No. 4,572,909. Further, U.S. Pat. No. 4,879,303 discloses that free base compositions that include microcrystalline cellulose and dicalcium phosphate as diluents excessively stick to the tablet punches during processing and are not suitable in making solid dosage forms for peroral administration. The patent teaches that the amlodipine besylate salt can be used to make solid dosage forms and those solid dosage forms can include microcrystalline cellulose and dibasic calcium phosphate. The microcrystalline cellulose is present at between about 62% (w/w) and about 76% (w/w) of the total dosage form composition. Subsequently, U.S. Patent Application No. 2003/0022922 discloses that to reduce the stickiness of the tablet, amlodipine free base should be incorporated into the tablet composition in the form of particulates having an average particle size of 150-350 μm; and a preferred excipient is a combination of calcium phosphate and microcrystalline cellulose. A capsule dosage form also is disclosed in—this patent application as containing amlodipine base, microcrystalline cellulose, predried potato starch, and magnesium stearate. The microcrystalline cellulose makes up approximately 74% (w/w) of the capsule dosage form.

Amlodipine is highly hygroscopic and absorbs moisture, which leads to degradation. One of the major routes of degradation is via the catalytic oxidative process, which is pH dependent. The major related substances produced are 13-ethyl 5-methyl(4RS)4-(2-chlorophenyl)methyl [[2-[[2-(methylcarbamoyl)benzoyl]amino]ethoxy]methyl]-1,4-dihydropyridine-3,5 dicarboxylate (“Impurity B”); 13-ethyl methyl [(2-aminoethoxy)methyl] (2-chlorophenyl)methylpyridine-3,5 dicarboxylate (“Impurity D”); and 13-ethyl 5-methyl (4RS)4-(2-chlorophenyl)-2[[2-(1,3-dioxo-dihydro-2H-isoindolyl)ethoxy]methyl]methyl-1,4-dihydropyridine-3,5 dicarboxylate (“Impurity A”), along with some unknown impurities. Being an unstable compound, amlodipine requires well-directed stability approaches to formulate pharmaceutical compositions with reasonable stability.

Benazepril is [S—(R*,R*)]-3-[[1-(ethoxycarbonyl)-3-phenylpropyl]amino]-2,3,4,5-tetrahydro-2-oxo-1H-1-benazepril-1-acetic acid. It is sold commercially in the form of the hydrochloride salt under the trademarks LOTENSIN® or CIBACEN® as an antihypertensive. Benazeprilat is the diacid form of benazepril formed by cleavage of the ester group, and is the active metabolite of benazepril. Benazepril and benazeprilat may be administered in free or pharmaceutically acceptable salt form. The therapy using benazepril or a pharmaceutically acceptable salt thereof or benazeprilat ranges from 10 mg to 80 mg per day.

Fixed dose combinations of amlodipine and benazepril are being marketed under the trade name Lotrel®. Corresponding amounts of the active ingredients are 2.5 mg of amlodipine and 10 mg of benazepril, 5 mg of amlodipine and 10 mg of benazepril, and 5 mg of amlodipine and 20 mg of benazepril, the amounts of amlodipine corresponding to the free base and the amounts of benazepril corresponding to the hydrochloride. This combination is indicated for the treatment of hypertension.

WO 02/49645 discloses that benazepril and amlodipine are physically incompatible. Hence, if incorporated into a single dosage form they must be kept physically separated. This may be accomplished in any of the myriad ways known in the art, such as bi-layered tablets, coated pellets of one agent incorporated into a tablet of the other, separately coated pellets of each agent in a capsule or tablet, coated pellets of one agent in capsule together with powder of the other agent, each agent microencapsulated separately and then blended together for use in a tablet or capsule, etc.

Present invention provides a stable solid dosage form comprising amlodipine and pharmaceutically acceptable salts thereof, and benazepril and pharmaceutically acceptable salts thereof.

### SUMMARY OF THE INVENTION

In one general aspect there is provided a stable solid dosage form. The dosage form includes (a) a first component comprising amlodipine or pharmaceutically acceptable salts thereof, and microcrystalline cellulose, wherein the component is substantially free of dicalcium phosphate; and (b) a second component comprising benazepril or pharmaceutically acceptable salts thereof, wherein the solid dosage form comprises less than about 0.2% concentration (w/w) of Impurity D after three months at 40° C. and 75% RH.

Embodiments of the present invention may include one or more of the following features. For example, the first component may include mannitol and wherein the solid dosage form comprises less than about 0.3% concentration (w/w) of Impurity D after three month at 40° C. and 75% RH.

The first component may include more than about 60% (w/w) of microcrystalline cellulose. The solid dosage form may include less than about 2% concentration (w/w) of total impurity after three month at 40° C. and 75% RH.

The dosage form may further include one or more pharmaceutically inert excipients selected from the group consisting of diluents, binders, desiccants, disintegrants, coloring agents, flavoring agents, stabilizers, surfactants, lubricants/glidants, plasticizers and preservatives.

The solid dosage form may be a tablet or a capsule. The tablet may be in the form of bilayer tablet or compression coated tablet.

In another general aspect there is provided a process for the preparation of a stable solid dosage form. The process includes (a) blending an effective amount of amlodipine, and one or more pharmaceutically inert excipients to form a first component, and optionally granulating and/or compressing the blend; (b) blending an effective amount of benazepril, and one or more pharmaceutically inert excipients to form a second component and optionally granulating and/or compressing the blend; and (c) blending the two components to form a solid dosage form.

The granulation may be carried out by wet granulation. The wet granulation may be carried out with a granulating fluid or solution/dispersion of binder. The granulation may also be carried out by dry granulation. The dry granulation may be carried out by a roller compactor or slugging.

In another general aspect there is provided a method of treating hypertension, chronic stable angina, or vasospastic angina in a mammal. The method includes administering to the said mammal a solid dosage form comprising: (a) a first component comprising amlodipine or pharmaceutically acceptable salts thereof, and microcrystalline cellulose, wherein the component is substantially free of dicalcium phosphate; and (b) a second component comprising benazepril or pharmaceutically acceptable salts thereof, wherein the solid dosage form has less than about 0.2% concentration (w/w) of Impurity D after three months at 40° C. and 75% RH.

### DETAILED DESCRIPTION OF THE INVENTION

The prior art literature discloses the use of dicalcium phosphate as one of the preferred excipients for amlodipine formulations. The inventors have discovered that the presence of dicalcium phosphate in the amlodipine formulation triggers the degradation of amlodipine, which is more pronounced at a pH below 6. Hence, removal of dicalcium phosphate form the composition provides a more stable pharmaceutical compositions of amlodipine.

In our attempts to stabilize amlodipine in solid dosage forms we discovered that stability may be improved by replacing dicalcium phosphate with microcrystalline cellulose and mannitol.

The term “stable” as used herein refers to the chemical stability of amlodipine in solid dosage forms and indicates the presence of less than 2% w/w of related substances when stored at 40° C. and 75% percent relative humidity for three month. The stability is measured using HPLC to measure the presence of related substances.

Amlodipine as used herein is the free base or besylate salt and can be of any form including, anhydrous, hydrous, crystalline form I, crystalline form II, amorphous form, and mixtures thereof.

Pharmaceutically acceptable salts of benazepril include inorganic or organic acids, such as strong mineral acids, for example hydrohalic, e.g. hydrochloric or hydrobromic acid; sulfuric, phosphoric, nitric or perchloric acid; aliphatic or aromatic carboxylic or sulfonic acids, e.g. formic, acetic, propionic, succinic, glycolic, lactic, malic, tartaric, gluconic, citric, ascorbic, maleic, fumaric, hydroxymaleic, pyruvic, phenylacetic, benzoic, 4-aminobenzoic, anthranilic, 4-hydroxybenzoic, salicylic, 4-aminosalicyclic, pamoic, nicotinic; methanesulfonic, ethanesulfonic, hydroxyethanesulfonic, benzenesulfonic, p-toluenesulfonic, naphthalenesulfonic, sulfanilic or cyclohexylsulfamic acid.

The term “dicalcium phosphate” as used herein includes anhydrous calcium phosphate, anhydrous dicalcium phosphate, dibasic calcium phosphate as well as hydrates and solvates thereof. Dicalcium phosphate is normally used as a diluent.

The term “substantially free” as used herein refers to the use of dicalcium phosphate in a concentration less than that used as a diluent. Microcrystalline cellulose is a white, odorless, tasteless, free flowing powder, and is widely accepted in the pharmaceutical industry as a universal diluent. It is purified; partially depolymerized alpha cellulose derived from purified specialty grades of wood pulp. There are various grades which differ in bulk density, particle size, and moisture content. Some of the commercially available grades of microcrystalline cellulose are Avicel®, Vivapur® and Tabulos®. When used without mannitol, the amount of microcrystalline cellulose is increased relative to the prior art, e.g., greater than 80% (w/w) and, more particularly, greater than 90% (w/w). When used with mannitol, the amount of microcrystalline cellulose is greater than about 60% (w/w).

Mannitol is a naturally occurring sugar alcohol having a cool taste and 50% sweetness compared to sucrose. It is non-hygroscopic, chemically inert and does not undergo the Maillard reaction, and therefore does not discolor in the presence of free amines. Mannitol is available as powder and free flowing granules, and is used widely in pharmaceutical preparations. The granular form is particularly useful in direct compression technique of preparing tablets. Some of the commercial grades are Mannogem®, Pearlitol® and Partech M®. The concentration of mannitol may vary from about 5% to about 80%, in particular it may vary from 20% to 60% by weight of the total uncoated tablet weight.

The term “solid dosage form” as used herein includes conventionally used dosage forms such as tablet, capsule and the like.

The term “pharmaceutically inert excipient” as used herein includes substances known in the art as diluents, binders, desiccants, disintegrants, coloring agents, flavoring agents, stabilizers, surfactants, lubricants/glidants, plasticizers and preservatives for pharmaceutical compositions. The excipients are selected based on the desired physical aspects of the final tablets; e.g., obtaining a tablet with desired hardness and friability, being rapidly dispersible and easily swallowed, etc. Further, the inert excipients may be so selected as to provide slow and/or controlled release of the amlodipine from the tablets.

Examples of disintegrants include sodium starch glycolate, croscarmellose sodium, crospovidone, low substituted hydroxypropyl cellulose, and mixtures thereof.

Examples of binders include methyl cellulose, hydroxypropyl cellulose, hydroxypropyl methylcellulose, polyvinylpyrrolidone, gelatin, gum arabic, ethyl cellulose, polyvinyl alcohol, pullulan, pregelatinized starch, agar, tragacanth, sodium alginate, and mixtures thereof.

Examples of diluents include cellulose powdered, dextrates, dextrins, dextrose excipients, fructose, kaolin, lactitol, lactose, mannitol, sorbitol, starch, starch pregelatinized, sucrose, sugar compressible, sugar confectioners, and mixtures thereof.

Examples of lubricants and glidants include magnesium stearate, colloidal anhydrous silica, stearic acid, magnesium stearate, calcium stearate, talc, hydrogenated castor oil, sucrose esters of fatty acid, microcrystalline wax, yellow beeswax, white beeswax, and mixtures thereof.

Examples of desiccants include colloidal silicon dioxide, silicon dioxide and the like.

Examples of surfactants include both non-ionic and ionic (cationic, anionic and zwitterionic) surfactants suitable for use in pharmaceutical compositions. These include polyethoxylated fatty acids and its derivatives, for example polyethylene glycol 400 distearate, polyethylene glycol-20 dioleate, polyethylene glycol 4-150 mono dilaurate, polyethylene glycol-20 glyceryl stearate; alcohol-oil transesterification products, for example polyethylene glycol-6 corn oil; polyglycerized fatty acids, for example polyglyceryl-6 pentaoleate; propylene glycol fatty acid esters, for example propylene glycol monocaprylate; mono and diglycerides, for example glyceryl ricinoleate; sterol and sterol derivatives, for example sitosterol; sorbitan fatty acid esters and its derivatives, for example polyethylene glycol-20 sorbitan monooleate, sorbitan monolaurate; polyethylene glycol 8 alkyl ether or phenols, for example polyethylene glycol-20 cetyl ether, polyethylene glycol 10-100 nonyl phenol; sugar esters, for example sucrose monopalmitate; polyoxyethylene polyoxypropylene block copolymers known as “poloxamer”; ionic surfactants, for example sodium caproate, sodium glycocholate, soy lecithin, sodium stearyl filmarate, propylene glycol alginate, octyl sulfosuccinate disodium, palmitoyl carnitine; and mixtures thereof.

Examples of plasticizers include polyethylene glycol, triethyl citrate, triacetin, diethyl plithalate, dibutyl sebacate and mixtures thereof.

Examples of stabilizers include antioxidants, buffers, alkalizers, chelating agents and mixtures thereof.

Examples of coloring agents include any FDA approved colors for oral use.

According to one embodiment, there is provided a process for the preparation of stable solid dosage form comprising:

(a) one component comprising amlodipine involving the steps of:
i) blending an effective amount of amlodipine, and one or more pharmaceutically inert excipients,
   (b) a second component comprising benazepril or pharmaceutically acceptable salts thereof and a carrier,
i) blending an effective amount of benazepril, and one or more pharmaceutically inert excipients,
ii) granulating and compressing the blend into suitable size tablet,
iii) optionally film coating the tablet,
   (c) filling the benazepril tablets and amlodipine blend into capsules.
   In another embodiment, there is provided a process for the preparation of stable solid dosage form comprising:
   (a) one component comprising amlodipine involving the steps of:
i) blending an effective amount of amlodipine, and one or more pharmaceutically inert excipients,
ii) granulating and compressing the blend into suitable size tablet,
iii) optionally film coating the tablet,
   (b) a second component comprising benazepril or pharmaceutically acceptable salts thereof and a carrier,
i) blending an effective amount of benazepril, and one or more pharmaceutically inert excipients,
   (c) filling the amlodipine tablets and benazepril blend into capsules.

In another embodiment, there is provided of a process for the preparation of a stable solid dosage form comprising amlodipine and benazepril or pharmaceutically acceptable salts thereof comprising the steps of:

a) coating inert core with a drug layer comprising amlodipine and

b) coating separate inert cores with a drug layer comprising benazepril,

c) filling the beads of step a) and b) into capsules.

In another embodiment, there is provided a process for the preparation of a stable solid dosage form comprising amlodipine and benazepril or pharmaceutically acceptable salts thereof comprising the steps of:

a) blending amlodipine and one or more pharmaceutically inert excipients;

b) optionally granulating the blend,

c) lubricating the blend or granules,

d) compressing into suitable size solid dosage form,

e) coating the core with a layer of inert excipients,

f) dispersing or dissolving the benazepril and other inert excipients in a solvent,

g) coating the core of step e) with drug layer.

In another embodiment, there is provided a process for the preparation of a pharmaceutical composition comprising amlodipine and benazepril or pharmaceutically acceptable salts thereof comprising the steps of:

a) blending benazepril and one or more pharmaceutically inert excipients;

b) optionally granulating the blend,

c) lubricating the blend or granules,

d) compressing into suitable size solid dosage form,

e) coating the core with a layer of inert excipients,

f) dispersing or dissolving the amlodipine and other inert excipients in a solvent,

g) coating the core of step e) with drug layer.

In another embodiment, there is provided a process for the preparation of a pharmaceutical composition comprising amlodipine and benazepril or pharmaceutically acceptable salts thereof comprising the steps of

i) preparation of amlodipine granules

a) blending amlodipine and one or more pharmaceutically inert excipients;

b) optionally granulating the blend,

c) lubricating the blend or granules,

ii) preparation of benazepril granules

a) blending benazepril and one or more pharmaceutically inert excipients;

b) optionally granulating the blend,

c) lubricating the blend or granules,

iii) compressing the above two granules into a bilayer tablet.

Granulation may be carried out by wet granulation or dry granulation techniques. Coating may be performed by applying one or more film forming polymers, with or without other pharmaceutically inert excipients, as a solution/suspension using any conventional coating technique known in the art, such as spray coating in a conventional coating pan or fluidized bed processor, or dip coating.

The following examples illustrate the invention but should not be construed as limiting the scope of the invention.

### EXAMPLE 1 & 2

### Capsule Comprising Amlodipine Besylate Powder Blend and Benazepril Hydrochloride Tablet

Wt/capsule (mg)

| | |
|---|---|
| Example 1 | Example 2 |

Amlodipine Powder Blend

| | | |
|---|---|---|
| Amlodipine besylate eq. | 10 | — |
| to amlodipine | | |
| Amlodipine besylate | — | 10 |
| eq. to amlodipine | | |
| (Micronised) | | |
| Microcrystalline | 117.11 | 117.11 |
| cellulose | | |
| Mannitol | 63.00 | 63.00 |
| Sodium starch glycolate | 4.00 | 4.00 |
| Magnesium stearate | 2.00 | 2.00 |

Benazepril Hydrochloride tablet

| | | |
|---|---|---|
| Benazepril | 20.0 | 20.0 |
| Hydrochloride | | |
| Lactose monohydrate | 54.5 | 53.0 |
| Pregelatinized starch | 4.5 | 4.5 |
| Purified water | Qs | Qs |
| Microcrystalline | 13.0 | 13.0 |
| cellulose | | |
| Crospovidone | 2.5 | 3.5 |
| Colloidal silicone dioxide | 1.0 | 1.0 |
| Magnesium stearate | — | 0.5 |
| Hydrogenated castor oil | 4.5 | 4.5 |
| Opadry white | 2.5 | 2.5 |
| Core tablet weight | 100.0 | 100.0 |

Amlodipine besylate powder blend according to the composition given above was prepared by the following steps:
1. Amlodipine besylate and microcrystalline cellulose were passed through #44 mesh.
2. Mannitol and Sodium starch glycol were passed through #44 mesh.
3. Blends of step 1 & step 2 were loaded into blender and blended for 20 minutes.
4. Magnesium stearate was passed through #60 mesh and lubricated with blend of step 3 for 5 minutes.
   Benazepril hydrochloride tablets were prepared by using the following steps:
1. Benazepril Hydrochloride, lactose monohydrate and Pregelatinized starch were passed through sieve and mixed in Rapid mixer granulator.
2. The above blend was granulated with purified water and dried.
3. Dried granules were passed through sieve.
4. Microcrystalline cellulose, Crospovidone and Colloidal silicon dioxide were passed through sieve and blended with blend of step 3.
5. Hydrogenated castor oil and Magnesium stearate (for example 10) were passed through #60 meshes and the blend of step 4 was lubricated.
6. The blend of step 5 was compressed into suitable size tablet.
7. Core tablets were coated with Opadry.

### Capsule

The amlodipine powder blend and benazepril tablet were filled into capsules.

The capsules obtained above were subjected to stability evaluation at 40° C. and 75% relative humidity. Initially, after one month, the tablets were evaluated for the presence of impurities using HPLC. The results of this measurement are listed as percentage (w/w) related substances in Table 1.

TABLE 1
Results of stability evaluation of capsule comprising amlodipine
besylate powder blend and benazepril hydrochloride tablet
(Examples 1&2) as percentage (w/w) related substances,
at 40° C. and 75% relative humidity.

| Related | | | | | |
|---|---|---|---|---|---|
| substance | Example 1 | | | Example 2 | |
| for | Conc. % (w/w) | | | | |
| Amlodipine | Initial | 1 M* | 3 M* | Initial | 1 M* |
| Impurity D | ND** | ND** | 0.010 | ND** | ND** |
| Impurity B | 0.046 | 0.043 | 0.008 | 0.052 | 0.045 |
| Impurity A | 0.009 | 0.001 | 0.021 | ND** | ND** |
| Highest | 0.071 | 0.079 | 0.009 | 0.076 | 0.073 |
| Unknown | | | | | |
| Total | 0.084 | 0.089 | 0.009 | 0.088 | 0.100 |
| Unknown | | | | | |
| Total | 0.393 | 0.488 | 1.306 | 0.537 | 0.703 |
| Related | | | | | |
| substance of | | | | | |
| the solid | | | | | |
| dosage form | | | | | |

*Month,
**Not Detected

### In Vitro Dissolution Study

In vitro release of amlodipine and benazepril as per composition of Example 1 and 2 was carried out in 500 ml 0.01 N HCl in USP type I apparatus at 100 rpm. The results are shown in Table 2.

TABLE 2
Comparative drug release profile of amlodipine and benazepril
formulation and Lotrel ® Capsules.

| | | | |
|---|---|---|---|
| | | | Cumulative |
| | Cumulative percentage | Cumulative percentage | percentage |
| | of drug released from | of drug released from | of drug |
| Time | the formulation of | the formulation of | released from |
| (min) | example 1 | example 2 | Lotrel ® |

Amlodipine Part

| | | | |
|---|---|---|---|
| 10 | 91 | 94 | 97 |
| 15 | 96 | 95 | 97 |
| 20 | 97 | 96 | 97 |
| 30 | 98 | 95 | 96 |
| 45 | 98 | 95 | 97 |

Benazepril Part

| | | | |
|---|---|---|---|
| 10 | 69 | 93 | 94 |
| 15 | 99 | 96 | 99 |
| 20 | 99 | 97 | 100 |
| 30 | 99 | 97 | 100 |
| 45 | 99 | 97 | 100 |

### EXAMPLE 3

### Compression Coated Tablets Comprising Benazepril Hydrochloride Inner Tablet and Amlodipine Outer Layer Tablet were Prepared as Per Composition Given Below

Wt/capsule (mg)
Example 11

Benazepril Hydrochloride tablet

| | |
|---|---|
| Benazepril | 20.0 |
| Hydrochloride | |
| Lactose monohydrate | 51.5 |
| Pregelatinized starch | 4.5 |
| Purified water | Qs |
| Macrocrystalline | 13.0 |
| cellulose | |
| Crospovidone | 2.5 |
| Colloidal silicone dioxide | 1.0 |
| Magnesium stearate | 0.5 |
| Hydrogenated castor oil | 4.5 |
| Core tablet weight | 100.0 |
| Opadry white | 2.5 |

Amlodipine Blend

| | |
|---|---|
| Amlodipine besylate | 13.888 |
| Microcrystalline | 238.112 |
| cellulose | |
| Mannitol | 126.0 |
| Sodium starch glycolate | 12.0 |
| Colloidal silicon dioxide | 4.0 |
| Magnesium stearate | 6.0 |
| Weight | 400.0 |

Benazepril hydrochloride tablets were prepared by using the following steps:
1. Benazepril hydrochloride, lactose monohydrate and pregelatinized starch were passed through sieve and mixed in rapid mixer granulator.
2. The above blend was granulated with purified water and dried.
3. Dried granules were passed through sieve.
4. Microcrystalline cellulose, crospovidone and colloidal silicon dioxide were passed through sieve and blended with blend of step 3.
5. Hydrogenated castor oil, colloidal silicon dioxide and magnesium stearate were passed through sieve and the blend of step 4 was lubricated.
6. The blend of step 5 was compressed into suitable size tablet.
7. Core tablets were coated with Opadry.
   Amlodipine besylate powder blend was prepared by using the following steps:
   1. Amlodipine besylate and a part of microcrystalline cellulose were passed through sieve.
   2. Mannitol, a part of microcrystalline cellulose, colloidal silicon dioxide and sodium starch glycol were passed through sieve.
   3. Blends of step 1 & step 2 were loaded into blender and blended for 20 minutes.
   4. Magnesium stearate was passed through sieve and lubricated with blend of step 3 for 5 minutes.

Amlodipine powder blend was compressed over benazepril hydrochloride tablet to produce a compression coated tablet with benazepril hydrochloride as inner tablet and amlodipine as outer layer.

While the present invention has been described in terms of its specific embodiments, certain modifications and equivalents will be apparent to those skilled in the art and are included within the scope of the present invention.

We claim:

1. A stable solid dosage form comprising:
(a) a first component comprising amlodipine or pharmaceutically acceptable salts thereof, and microcrystalline cellulose, wherein the component is substantially free of dicalcium phosphate; and
(b) a second component comprising benazepril or pharmaceutically acceptable salts thereof, wherein the solid dosage form comprises less than about 0.2% concentration (w/w) of Impurity D after three months at 40° C. and 75% RH.

2. The stable solid dosage form according to claim 1, wherein the first component further comprises mannitol and wherein the solid dosage form comprises less than about 0.3% concentration (w/w) of Impurity D after three month at 40° C. and 75% RH.

3. The stable solid dosage form according to claim 1, wherein the first component comprises more than about 60% (w/w) of microcrystalline cellulose.

4. The stable solid dosage form according to claim 1, wherein the solid dosage form comprises less than about 2% concentration (w/w) of total impurity after three month at 40° C. and 75% RH.

5. The stable solid dosage form according to 1, wherein the dosage form further comprises one or more pharmaceutically inert excipients selected from the group consisting of diluents, binders, desiccants, disintegrants, coloring agents, flavoring agents, stabilizers, surfactants, lubricants/glidants, plasticizers and preservatives.

6. The stable solid dosage form according to claim 1, wherein the solid dosage form is a tablet or a capsule.

7. The stable dosage form according to claim 6, wherein the tablet is in the form of bilayer tablet or compression coated tablet.

8. A process for the preparation of a stable solid dosage form, the process comprising:
(a) blending an effective amount of amlodipine, and one or more pharmaceutically inert excipients to form a first component, and optionally granulating and/or compressing the blend;
(b) blending an effective amount of benazepril, and one or more pharmaceutically inert excipients to form a second component and optionally granulating and/or compressing the blend; and
(c) blending the two components to form a solid dosage form.

9. The process according to claim 10, wherein the granulation is carried out by wet granulation.

10. The process according to claim 11, wherein the wet granulation is carried out with a granulating fluid or solution/dispersion of binder.

11. The process according to claim 10, wherein the granulation is carried out by dry granulation.

12. The process according to claim 13, wherein the dry granulation is carried out by a roller compactor or slugging.

13. A method of treating hypertension, chronic stable angina, or vasospastic angina in a mammal, the method comprising administering to the said mammal a solid dosage form comprising:
(a) a first component comprising amlodipine or pharmaceutically acceptable salts thereof, and microcrystalline cellulose, wherein the component is substantially free of dicalcium phosphate; and
(b) a second component comprising benazepril or pharmaceutically acceptable salts thereof, wherein the solid dosage form has less than about 0.2% concentration (w/w) of Impurity D after three months at 40° C. and 75% RH.
